(19) United States
(12) Patent Application Publication
(10) Pub. No.: US 2010/0114227 A1
(43) Pub. Date: May 6, 2010

(54) Systems and Methds for Use by an Implantable Medical Device for Controlling Vagus Nerve Stimulation Based on Heart Rate Reduction Curves and Thresholds to Mitigate Heart Failure

(75) Inventor: Martin Cholette, Acton, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/261,924

(22) Filed: Oct. 30, 2008

(51) Int. Cl. A61N 1/365

(52) U.S. Cl. 607/17

(57) ABSTRACT

Systems and techniques are provided for controlling vagus nerve stimulation (VNS) delivered by an implantable medical device for mitigating heart failure in a patient. In one mode, VNS therapy is set to levels just below a heart rate reduction threshold so as to deliver VNS near the highest stimulation levels that can be achieved without reducing patient heart rate. In this manner, a maximum level of heart failure mitigation can be achieved via VNS therapy without incurring the potentially adverse consequences of inducing bradycardia within the patient. In another mode, VNS therapy is instead controlled to deliver VNS above the threshold so as to mitigate heart failure while also selectively reducing heart rate, as may be appropriate in patients susceptible to cardiac ischemia. A controlled heart rate reduction curve may additionally or alternatively be determined for use in achieving target amounts of heart rate reduction.

### FIELD OF THE INVENTION

The invention relates to implantable medical devices equipped to deliver vagus nerve stimulation (VNS) to mitigate heart failure and to techniques for controlling VNS.

### BACKGROUND OF THE INVENTION

Heart failure is a debilitating disease in which abnormal function of the heart leads in the direction of inadequate blood flow to fulfill the needs of the tissues and organs of the body. Typically, the heart loses propulsive power because the cardiac muscle loses capacity to stretch and contract. Often, the ventricles do not adequately eject or fill with blood between heartbeats and the valves regulating blood flow become leaky, allowing regurgitation or back-flow of blood. The impairment of arterial circulation deprives vital organs of oxygen and nutrients. Fatigue, weakness and the inability to carry out daily tasks may result.

Not all heart failure patients suffer debilitating symptoms immediately. Some may live actively for years. Yet, with few exceptions, the disease is relentlessly progressive. As heart failure progresses, it tends to become increasingly difficult to manage. Even the compensatory responses it triggers in the body may themselves eventually complicate the clinical prognosis. For example, when the heart attempts to compensate for reduced cardiac output, it adds muscle causing the ventricles (particularly the left ventricle) to grow in thickness in an attempt to pump more blood with each heartbeat. This places a still higher demand on the heart's oxygen supply. If the oxygen supply falls short of the growing demand, as it often does, further injury to the heart may result. The additional muscle mass may also stiffen the heart walls to hamper rather than assist in providing cardiac output. A particularly severe form of heart failure is congestive heart failure (CHF) wherein the weak pumping of the heart leads to build-up of fluids in the lungs and other organs and tissues.

One promising technique for mitigating heart failure is vagus nerve stimulation (VNS), wherein stimulate suitable branches of the vagus nerve are selectively stimulated. See, e.g., “Chronic Vagal Stimulation Exerts its Beneficial Effects on the Failing Heart Independently of its Anti-Beta-Adrenergic Mechanism,” Li et al., Circulation 2004;110(17 Supp.)—Abstract No. 396, and “Vagal Nerve Stimulation Markedly Improves Long-Term Survival After Chronic Heart Failure in Rats,” Li et al., Circulation 2004;109:120-124. VNS is believed to mitigate heart failure by counteracting parasympathetic withdrawal, sympathetic over-activation (i.e. catecholamine poisoning) and cardiac inflammatory activation. See, e.g., “Vagal stimulation markedly suppresses arrhythmias in conscious rats with chronic heart failure after myocardial infarction,” Zheng et al., Conf Proc IEEE Eng Med Biol Soc. 2005;7:7072-7075.

Heretofore, at least some techniques for mitigating heart failure via vagus nerve stimulation (VNS) operate to reduce the heart rate of the patient to below the customary resting heart rate of the patient, i.e. the VNS techniques induce bradycardia. See, e.g., U.S. Pat. No. 6,473,644 to Terry et al. However, the induction of bradycardia is not necessarily desirable in all heart failure patients and, indeed, can often be counterproductive. The reduction in heart rate can result in a corresponding reduction in cardiac output. Usually, it is instead desirable to increase cardiac output within heart failure patients to, for example, reduce the risk of pulmonary edema. Moreover, to compensate for the loss of cardiac output due to reduced heart rate, the heart of the patient may need to beat more vigorously during each contraction to improve stroke volume, which can further exacerbate heart failure by, e.g., significantly and dangerously enlarging the myocardium of the left ventricle.

Hence, it would be desirable to provide improved techniques for controlling VNS so as to mitigate heart failure without unnecessarily reducing patient heart rate, and it is to this end that aspects of the invention are directed.

It should be noted that, within at least some heart failure patients, a reduction of heart rate achieved via VNS can be beneficial, particularly within patients whose heart rate is high and who are susceptible to cardiac ischemia. Accordingly, it is also desirable to provide improved techniques for controlling VNS so as to mitigate heart failure while additionally achieving a controllable amount of heart rate reduction, and it is to this end that other aspects of the invention are directed.

Still further aspects of the invention are directed to implementing the improved VNS techniques within implantable medical devices, such as pacemakers, implantable cardioverter/defibrillators (ICDs) or stand-alone VNS controllers.

### SUMMARY OF THE INVENTION

In an exemplary embodiment, a method for controlling VNS is provided for use with an implantable medical device for implant within a patient, such as a suitably-equipped pacemaker, ICD or stand-alone VNS controller. Briefly, the vagus nerve of the patient is stimulated in accordance with at least one adjustable VNS parameter, such as VNS pulse amplitude, while the heart rate of the patient is monitored. A threshold level for the VNS parameter is determined at which VNS begins to reduce heart rate. Herein, the threshold level is generally referred to herein as the “heart rate reduction threshold.” Further VNS therapy is then controlled based on the heart rate reduction threshold level.

In one exemplary therapy mode, denoted “Mode 1,” VNS therapy is controlled based on the heart rate reduction threshold so as to deliver VNS at or near the highest stimulation levels that can be achieved without reducing heart rate. In this manner, a maximum level of heart failure mitigation is achieved via VNS therapy without incurring the potentially adverse consequences of inducing a possible bradycardia. In another exemplary therapy mode, herein denoted “Mode 2,” VNS therapy is controlled based on the heart rate reduction threshold level so as to deliver VNS at a selected level above the threshold so as to mitigate heart failure while also reducing heart rate. In this manner, for patients in whom a reduction in heart rate might be beneficial (such as patients susceptible to cardiac ischemia), heart failure mitigation is achieved via VNS therapy while also reducing the heart rate. In still yet another embodiment, the heart rate of the patient is monitored while VNS therapy is delivered in Mode 1 to mitigate heart failure. If the heart rate of the patient increases above an acceptable “tolerance threshold” rate, VNS therapy is then switched to Mode 2 to reduce patient heart rate, while also mitigating heart failure.

With regard to the heart rate reduction threshold, it has been found that the heart rate reducing properties of VNS are mediated by Type C small diameter unmyelinated vagal fibers. Meanwhile, the anti-inflammatory, sympatholytic properties of VNS are mediated by Type A & B large diameter, myelinated vagal fibers. The capture threshold of the Type C fibers exceeds that of both the Type A & B fibers. Accordingly, by setting the VNS parameters just below the aforementioned heart rate reduction threshold, the Type A & B vagus fibers are thereby activated or triggered, without also triggering the Type C fibers. Hence, for patients where heart rate reduction is unnecessary or counterproductive, VNS therapy is delivered to achieve the greatest amount of stimulation of the Type A & B vagus fibers to mitigate heart failure, without also triggering the Type C fibers that reduce heart rate. For any patients who might instead benefit from a reduced heart rate, VNS therapy can be delivered above the heart rate reduction threshold to trigger the Type A & B vagus fibers while also triggering at least some of the Type C fibers so as to reduce heart rate. The initial determination of the heart rate reduction threshold is thereby important in either case, as it allows for controlling VNS relative to the threshold.

In an illustrative example, the heart rate reduction threshold level is determined for a particular VNS parameter, such as VNS pulse amplitude, by incrementally adjusting the VNS parameter until a predetermined amount of heart rate reduction is detected. For example, the VNS pulse amplitude can be incremented until at least a three beat-per-minute (bpm) reduction in patient heart rate is detected. The pulse amplitude value that triggered the three-bpm reduction is then designated as the heart rate reduction threshold for pulse amplitude. Thereafter, to deliver VNS within Mode 1 (i.e. without a reduction in heart rate), the pulse amplitude may be set to, e.g., 90% of the threshold value. To instead deliver VNS in Mode 2 (i.e. with a reduction in heart rate), the pulse amplitude may be set to some amount above the threshold value.

Additionally or alternatively, a “controlled heart rate curve” may be determined for the patient, which relates VNS pulse amplitude to the amount of heart rate reduction (if any). Using such a curve, VNS can be easily controlled to achieve heart failure therapy (via the activation of the Type A & B vagus fibers) in conjunction with a targeted amount of heart rate reduction (via activation of Type C vagus fibers.) The controlled heart rate curve can be determined, e.g., by continuing to increment the VNS pulse amplitude (even after the heart rate reduction threshold has been exceeded) so as to track heart rate reduction vs. VNS pulse amplitude, at least until some maximum acceptable level of heart rate reduction is reached, such as 25 bpm. Other VNS stimulation parameters that might be similarly exploited include pulse width, pulse frequency, the shape of the VNS pulse and, if burst VNS is employed, the applicable burst parameters. So long as the value of a given VNS parameter has some influence over whether VNS stimulation triggers Type C vagus fiber capture thresholds, then the parameter can have a corresponding controlled heart rate curve associated therewith.

In one particular example, the heart rate reduction threshold and the controlled heart rate curve are both determined for the patient. The controlled heart rate curve is exploited within Mode 2 to set the VNS parameters to achieve a preferred or targeted amount of heart rate reduction in that mode. In other examples, the heart rate reduction threshold is not explicitly determined for the patient. Rather, the implanted device instead just uses the controlled heart rate curve to set the VNS parameters to achieve a targeted amount of heart rate reduction (if any) within the patient. Also, note that the clinician programming the operation of the implanted device preferably specifies the particular VNS parameters to be exploited by the device in its various modes of operation and to specify any other needed parameters such as the “tolerance threshold” for the patient.

Exemplary system and method implementations are described below.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 illustrates pertinent components of an implantable medical system having a pacer/ICD equipped to control delivery of VNS to the cardiac branch of the vagus nerve of a patient based on the heart rate reduction threshold of the patient and/or based on a controlled heart rate curve determined for the patient;

FIG. 2 provides an overview of a general method performed by the system of FIG. 1 for determining the heart rate reduction threshold of the patient and for controlling delivery of VNS to the patient based thereon;

FIG. 3 illustrates an exemplary embodiment of the technique of FIG. 2 wherein the heart rate reduction threshold is exploited to deliver VNS to mitigate heart failure without a reduction in heart rate, by triggering only Type A & B fibers (i.e. Mode 1 operation);

FIG. 4 is a graph illustrating relative strength duration curves for Type A, B and C vagus fibers, which is exploited by the technique of FIG. 3 to trigger Type A & B fibers without triggering Type C fibers;

FIG. 5 illustrates another exemplary embodiment of the technique of FIG. 2 wherein the heart rate reduction threshold is exploited to deliver VNS to mitigate heart failure while also reducing heart rate reduction by triggering Type C fibers in addition to Type A & B fibers (i.e. Mode 2 operation);

FIG. 6 illustrates yet another exemplary embodiment of the technique of FIG. 2 wherein a heart rate tolerance threshold for the patient is exploited to control switching between Mode 1 and Mode 2;

FIG. 7 provides an overview of a general method performed by the system of FIG. 1 for determining a controlled heart rate curve for the patient and for controlling delivery of VNS to the patient based thereon;

FIG. 8 is a graph illustrating an exemplary controlled heart rate curve determined by the technique of FIG. 7;

FIG. 9 illustrates an exemplary embodiment of the technique of FIG. 8 wherein the controlled heart rate curve is determined for the patient for use in controlling delivery of VNS to the patient to achieve targeted heart rate reduction;

FIG. 10 is a simplified, partly cutaway view, illustrating the pacer/ICD of FIG. 1 along with a more complete set of exemplary pacing/sensing leads implanted in or on the heart of a patient, and also illustrating a vagus nerve stimulator; and

FIG. 11 is a functional block diagram of the pacer/ICD of FIG. 10, illustrating basic device circuit elements that provide cardioversion, defibrillation and/or pacing stimulation in four chambers of the heart and particularly illustrating components within the device for controlling delivery of VNS to the vagus nerve of the patient based on the heart rate reduction threshold and/or the controlled heart rate curve.

### DETAILED DESCRIPTION OF THE PREFERRED EMBODIMENTS

The following description includes the best mode presently contemplated for practicing the invention. This description is not to be taken in a limiting sense but is made merely to describe general principles of the invention. The scope of the invention should be ascertained with reference to the issued claims. In the description of the invention that follows, like numerals or reference designators are used to refer to like parts or elements throughout.

### Overview of Implantable Medical System

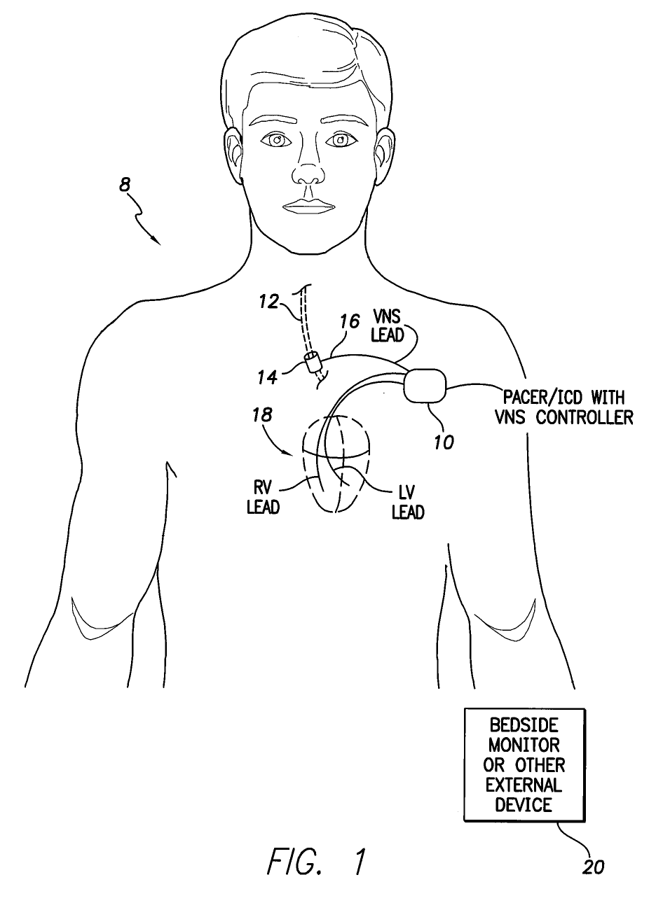

FIG. 1 illustrates an implantable medical system 8 capable of delivering vagus nerve stimulation (VNS) to the patient in which the system is implanted so as to, e.g., mitigate heart failure. To this end, a pacer/ICD 10 (or other suitable implantable medical device) delivers VNS via to one or more of the cardiac branches of the vagus nerve 12 of the patient (also generally referred to as the 10th cranial nerve.) VNS is delivered to a suitable branch of the vagus nerve by a vagus nerve stimulator 14 operating under the control of the pacer/ICD via control signals sent along a VNS lead 16. In particular, pacer/ICD 10 is equipped to control VNS based on a heart rate reduction threshold and/or a controlled heart rate curve so as to mitigate heart failure while selectively controlling patient heart rate. Details of these techniques are provided below.

Note that any of a variety of suitable neural stimulator devices and neural stimulation techniques may be employed within the system of FIG. 1 for actually delivering VNS to the vagus nerve of the patient. VNS devices and techniques are discussed, for example, in U.S. Pat. No. 6,934,583 to Weinberg, as well as in the aforementioned patent to Terry et al. Within FIG. 1, stylized representations of the vagus nerve stimulator and of one of the branches of the vagus nerve are shown. The actual size, location and shape of the vagus nerve of the patient and of the vagus nerve stimulator implanted therein may differ from that shown within practical implementations.

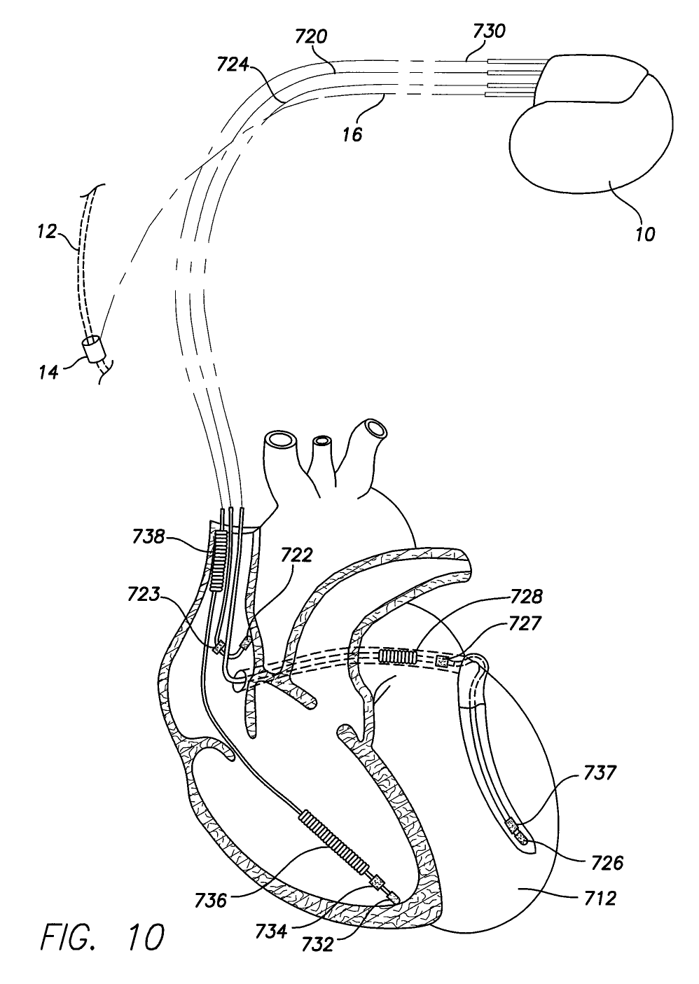

The implantable medical system also includes a set of cardiac pacing/sensing/shocking leads 18 for sensing cardiac signals, delivering pacing therapy, delivering cardioversion shocks, etc., also under the control of the pacer/ICD. By controlling VNS using a pacer/ICD, the patient may thereby derive additional benefit from the many features and functions of the pacer/ICD. However, it should be understood that VNS might instead be controlled by a stand-alone implantable VNS controller without the use of a pacer/ICD or its leads. Note also that leads 18 and the heart of the patient are shown only in a stylized form. A more thorough and anatomically correct illustration of the heart and the pacing/sensing/shocking leads is provided in FIG. 10 (described below).

FIG. 1 also illustrates a bedside monitor 20 (or other external device) for displaying and storing diagnostic information received from the implantable system, such as any diagnostic information pertaining to VNS. Information stored within the bedside monitor may be forwarded to remote systems (not shown in FIG. 1) for review by physicians or other medical professionals. For example, the bedside monitor may be directly networked with a centralized computing system, such as the HouseCall™ system or the Merlin.Net system of St. Jude Medical, for notifying the physician as to any issues arising with regard to VNS or other therapies delivered by the implantable system. Networking techniques for use with implantable medical systems are set forth, for example, in U.S. Pat. No. 6,249,705 to Snell, entitled “Distributed Network System for Use with Implantable Medical Devices.”

Hence, FIG. 1 provides an overview of an implantable medical system capable of delivering and controlling VNS and various cardiac pacing/sensing/shocking functions. Embodiments may be implemented that do not necessarily perform all of these functions or include all of these components.

### Overview of Heart Rate Reduction Threshold-Based VNS Techniques

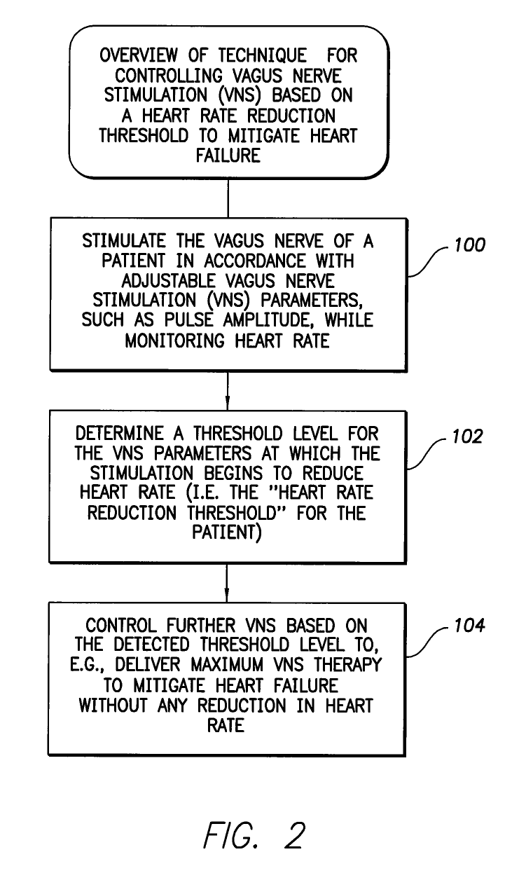

FIG. 2 provides a broad overview of heart rate reduction threshold-based VNS techniques that may be exploited by the pacer/ICD of FIG. 1 or other VNS controller device for mitigating heart failure. Briefly, beginning at step 100, a suitable branch of the vagus nerve of the patient is stimulated in accordance with adjustable VNS control parameters, such as pulse amplitude, while patient heart rate is monitored. By a suitable branch of the vagus nerve, it is meant that one or more branches of the vagus nerve are selected for stimulation that achieves some mitigation of heart failure when stimulated by, e.g., counteracting parasympathetic withdrawal, sympathetic over-activation and/or cardiac inflammatory activation. Otherwise conventional experimentation can be employed to identify suitable nerve branches and stimulation locations along those nerve branches. Typically, one or more of the cardiac branches of the vagus nerve are appropriate.

At step 102, the pacer/ICD determines a threshold level for the VNS parameters at which the stimulation begins to reduce heart rate. This is the aforementioned “heart rate reduction threshold.” For VNS pulse amplitude, the resulting threshold may be found to be 3.0 milliAmperes (mA) for a particular patient. That is, once the VNS pulse amplitude reaches 3.0 mA, patient heart rate begins to drop due to triggering or activation of Type C vagal fibers. (Exemplary techniques for detecting the heart rate reduction threshold of the patient are discussed below.) Note that each VNS parameter generally has a different heart rate reduction threshold. In use, the pacer/ICD is usually programmed to detect only the heart rate reduction threshold for one particular VNS parameter, such as pulse amplitude, which the pacer/ICD then adjusts to control VNS. However, in general, a heart rate reduction threshold might be determined for any or all adjustable VNS control parameters including VNS pulse amplitude, pulse frequency, pulse width (or duration), pulse shape (or morphology) or any of a variety of VNS burst stimulation parameters (such as burst duration or duty cycle.) So long as the value of a given VNS parameter has some influence over whether the VNS activates Type C vagus fibers, then the parameter can have a corresponding heart rate reduction threshold corresponding to the capture threshold of the Type C fibers. Otherwise routine experimentation can be performed to identify any particular VNS parameters that are most effective for the purposes of the invention. For example, within some patients, adjustment of pulse width rather than pulse amplitude might be a more effective technique for controlling VNS.

At step 104, further delivery of VNS within the patient is then controlled based on the detected threshold level to, e.g., deliver maximum VNS therapy to mitigate heart failure without any reduction in heart rate. As discussed above in the Summary, the heart rate reduction properties of VNS are mediated by Type C vagal fibers, whereas the anti-inflammatory, sympatholytic properties of VNS that mitigate heart rate are mediated by Type A & B vagal fibers. Since the capture threshold of Type C fibers exceeds that of Type A & B fibers, the heart rate reduction threshold generally serves to specify the capture threshold of the Type C fibers for the patient. Stimulation below the threshold triggers or activates only the Type A & B fibers, without triggering the Type C fibers. Hence, stimulation below the threshold serves to mitigate heart failure via activation of the Type A & B fibers without reducing heart rate. Stimulation above the threshold triggers at least some Type C fibers (along with the Type A & B fibers) to reduce heart rate while also mitigating heart failure. As such, the determination of the heart rate reduction threshold for the patient allows for precise control by the implantable system of the scope and effect of the VNS therapy to be delivered. Determination of the heart rate reduction threshold also allows for maximum heart failure mitigation therapy to be delivered without also reducing heart rate, which, as noted, can be problematic within at least some heart failure patients due to reduced cardiac output or other concerns.

Thus, FIG. 1 provides a broad overview of techniques for determining and exploiting the heart rate reduction threshold within a patient for use in controlling VNS. Note that the threshold may instead be referred to as a “bradycardia threshold” in the sense that stimulation above the threshold tends to cause the heart of the patient to beat at a rate below the rate at which it would otherwise be beating. However, this use of the term “bradycardia” is not intended to suggest that any pathological reduction in heart rate can or should be achieved via VNS.

### Mode 1: Heart Failure Mitigation Only

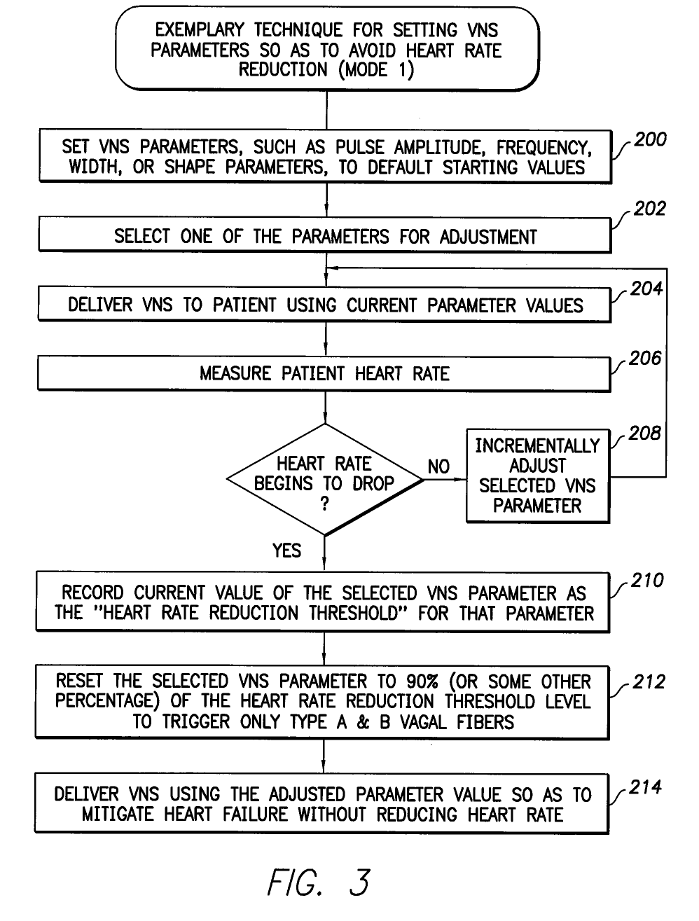

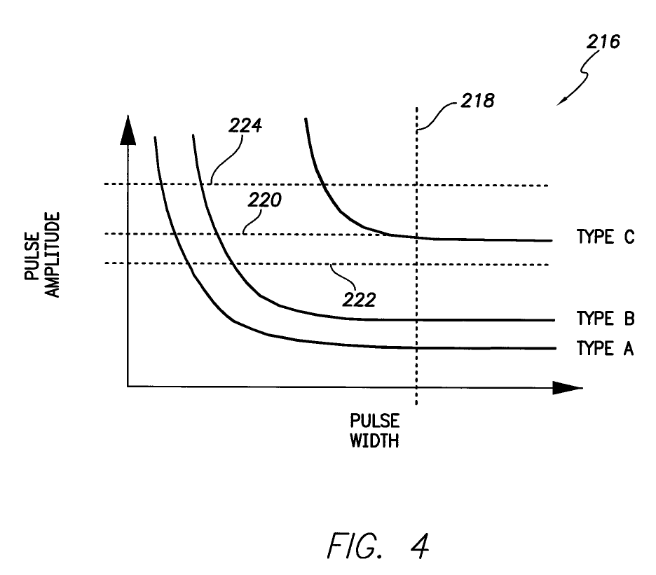

Turning now to FIGS. 3-4, an exemplary technique exploiting the heart rate reduction threshold will be described wherein stimulation below the threshold is employed to mitigate heart failure without reducing heart rate. Beginning at step 200 of FIG. 3, the pacer/ICD sets any programmable VNS parameters (such as pulse amplitude, frequency, width, shape parameters or burst parameters) to default starting values. At step 202, the pacer/ICD then selects one of the parameters for adjustment, such as pulse amplitude. This selection may be based on pre-programming of the pacer/ICD as specified, e.g., by the clinician's initial programming of the device.

At step 204, the pacer/ICD then delivers VNS to the patient using the current VNS control parameter values and, at step 206, measures patient heart rate. So long as the heart rate does not drop, the pacer/ICD incrementally adjusts the selected VNS parameter at step 208, while continuing to deliver VNS while monitoring heart rate. For pulse amplitude, the parameter value is increased at step 208 by some small amount, such as 0.5 mA, to increase the likelihood the VNS pulse will start to capture Type C fibers. Each iteration of steps 204-208 can be set to, e.g., in the range of ten to thirty seconds to allow time for the VNS stimulation to reduce heart rate (if Type C fibers are being triggered) and to allow the heart rate, if dropping, to stabilize at a new lower level. The average value of the stabilized heart rate may then be calculated. Note also that the actual heart rate of the patient need not be explicitly measured or calculated. Rather, related parameters such as R-R interval duration can instead be used. In one particular example, the last ten R-R intervals are averaged.

When a drop in heart rate is detected, then the pacer/ICD, at step 210, records the current value of the selected VNS parameter as the heart rate reduction threshold value for that parameter. This value also represents the Type C capture threshold for the selected parameter. Insofar as detecting a drop in heart rate, the pacer/ICD may be programmed to detect and measure any decrease in heart rate from its previous level (as determined during the initial iteration of steps 204-208) and to compare that decrease against a predetermined amount indicative of a significant or noticeable heart rate drop, such as a decrease of at least 3 bpm.

At step 212, the pacer/ICD then resets the selected VNS parameter to 90% (or some other percentage within a programmable range of, e.g., 25-95%) of the heart rate reduction threshold level to ensure triggering of only Type A & B vagal fibers. This new value for the parameter may be referred to as the “anti-HF only value” as it serves to trigger Type A & B fibers to mitigate heart failure without also reducing heart rate. Note that, with this particular technique, the capture thresholds of the Type A & B fibers are not determined and are not specifically known. However, by setting the VNS parameter to a high percentage of the heart rate reduction threshold value (e.g. 90%), it can be substantially assured that the VNS pulses will capture most of the Type A & B fibers (since it is known that Type C fibers have a still higher capture threshold) so as to facilitate heart failure mitigation. If the actual capture threshold for Type A & B fibers is known in advance (or can be otherwise ascertained), then the pacer/ICD can additionally take this information into account when setting the new value for the VNS parameter. Note also that if VNS pulses delivered at 90% of the rate reduction threshold level cause pain within the patient, the pulse amplitude (or other adjustable VNS parameter such as pulse width) can be reduced to eliminate such pain. For example, if it is found during an initial programming session that a 90% pulse amplitude setting causes pain within the patient, the VNS amplitude can be incrementally reduced (80%, 70%, 60%, etc.) until pain is eliminated. So long as the VNS amplitude is at least 25%, Type A fibers are captured to provide some degree of heart failure mitigation. Higher percentages are preferred so as to also capture Type B fibers (so long as there is no significant patient pain.)

Further VNS is then delivered at step 214 using the adjusted VNS parameter value so as to mitigate heart failure without reducing heart rate. Note that if the VNS parameter initially selected at step 202 is iterated through its entire range of acceptable values without triggering a drop in heart rate, then one of the other VNS parameters can instead be selected by the pacer/ICD for iterative adjustment. For example, if increases in VNS pulse amplitude do not trigger a drop in heart rate, then VNS pulse width may be iteratively increased. If no combination of parameters is found that triggers a drop in heart rate, then suitable warning signals may be generated and transmitted to the bedside monitor to notify the appropriate clinician that there might be a problem with the VNS stimulator within the patient. Also, note that the heart rate reduction threshold determined at step 210 for a given VNS parameter can be affected by the values of the other VNS parameters. This is shown by way of FIG. 4.

FIG. 4 illustrates pulse amplitude vs. pulse width (duration) capture threshold curves 216 for VNS stimulation for Type A, B and C vagal fibers, along with the aforementioned threshold values. The curves are provided for comparison only and so units are not specified along the axes of the graphs. As can be seen, for a given pulse width, a greater pulse amplitude is required to capture Type C fibers, as compared to Type A & B fibers. Moreover, the heart rate reduction threshold for pulse amplitude varies according to pulse width. Consider, for example, the pulse width specified by line 218. At that pulse width, the corresponding heart rate reduction threshold for pulse amplitude is specified by line 220. At a different value of pulse width, a different heart rate reduction threshold may arise (especially at shorter pulse widths.) Accordingly, when iterating the values of a selected VMS parameter using the technique of FIG. 3, it is best to hold the other values constant.

FIG. 4 also illustrates the “anti-HF only value” for VNS pulse amplitude based on pulse width 218. This value, which is set to 90% of threshold value 220, is identified by line 222. By delivering VNS with this particular combination of pulse amplitude and pulse width, Type A & B fibers are both captured, whereas the Type C fibers are not. FIG. 4 additionally illustrates that, when incrementally adjusting pulse amplitude, it is best to start with a relatively large pulse width (since very short pulse widths might not allow for capture of Type C fibers even at high pulse amplitudes.) Conversely, when incrementally adjusting pulse width, it is best to start with a relatively large pulse amplitude (since very low pulse amplitudes might not allow for capture of Type C fibers even at very long pulse durations.)

Mode 2: Heart Failure Mitigation with Heart Rate Reduction

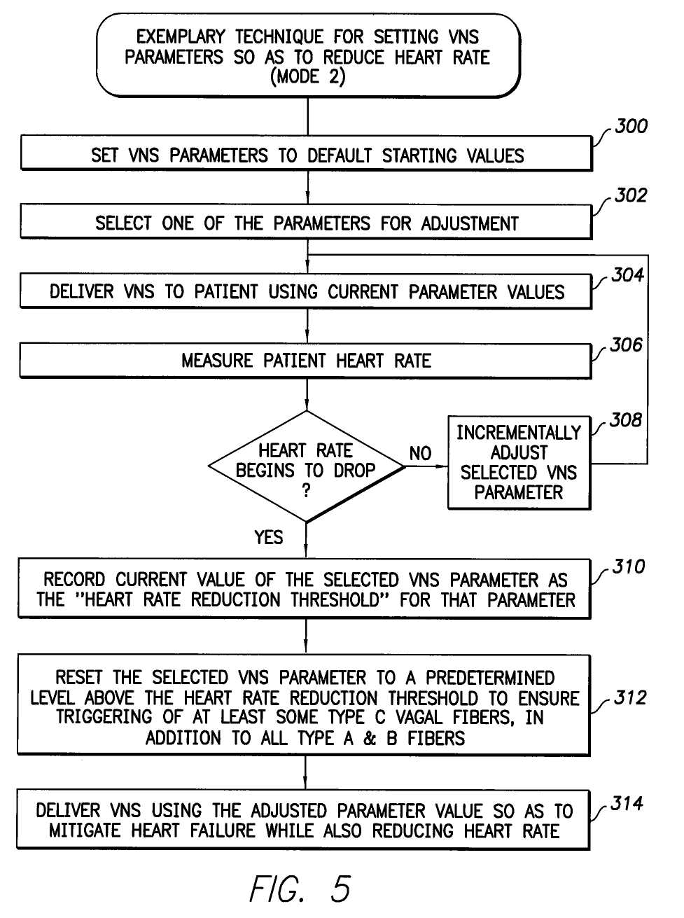

Turning now to FIG. 5, another exemplary technique that exploits the heart rate reduction threshold will be described. Some of the steps are the same or similar to those of FIG. 4 and hence those steps will only be described briefly. Beginning at step 300 of FIG. 5, the pacer/ICD sets the VNS parameters to default starting values and, at step 302, selects one of the parameters for adjustment. At steps 304 and 306, the pacer/ICD delivers VNS to the patient while tracking heart rate. If heart rate does not drop, the pacer/ICD incrementally adjusts the VNS parameter at step 308, then repeats steps 304 and 306. When a drop in heart rate is detected, the pacer/ICD records the heart rate reduction threshold value at step 310.

At step 312, the pacer/ICD resets the selected VNS parameter to some value above the heart rate reduction threshold level (such as 110% of that value) to ensure triggering of some Type C fibers in addition to the Type A & B vagal fibers. This new value for the parameter may be referred to as the “anti-HF plus HR reduction value.” Further VNS is then delivered at step 314 so as to mitigate heart failure while also reducing heart rate. The reduced heart rate may be beneficial in reducing the risk of cardiac ischemia. In order to achieve a targeted reduction in heart rate, the pacer/ICD may additionally determine and exploit a controlled heart rate reduction curve, which is described in detail below.

Referring again briefly to FIG. 4, an exemplary “anti-HF plus HR reduction value” is shown for VNS pulse amplitude by way of line 224. By delivering VNS at that pulse amplitude (and at the pulse width shown by line 218), at least some Type C fibers are recruited in addition to Type A & B fibers.

### Switching Between Mode 1 and Mode 2

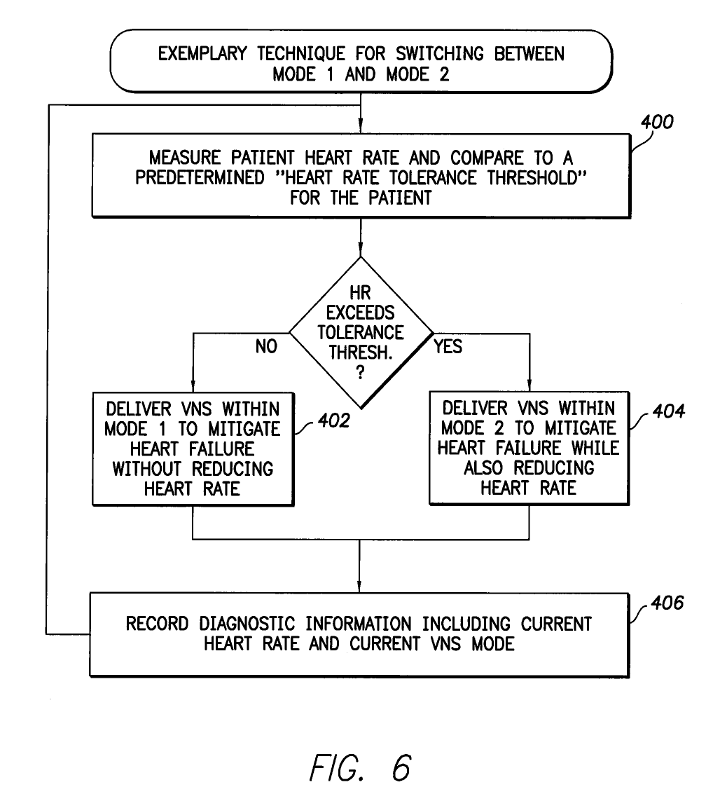

FIG. 6 illustrates an exemplary technique wherein Modes 1 and 2 are selectively activated based on the current heart rate of the patient. Beginning at step 400, the pacer/ICD measures patient heart rate and compares it to a predetermined “heart rate tolerance threshold” for the patient. Above this threshold, reduced perfusion is seen, muscle fatigue sets in and heart failure is exacerbated. The heart rate tolerance threshold is a programmable value specified by the clinician and may be set, e.g., in the range of 80 bpm-120 bpm. So long as the patient heart rate does not exceed the programmed tolerance threshold, VNS is delivered within Mode 1 at step 402 to mitigate heart failure without reducing heart rate. This is achieved, as already explained, by setting VNS parameters to values below their corresponding heart rate reduction thresholds. If heart rate exceeds the tolerance threshold, VNS is instead delivered within Mode 2 at step 404 to mitigate heart failure while also reducing heart rate. This is achieved, as also explained, by setting VNS parameters to values above their corresponding heart rate reduction thresholds. A predetermined controlled heart rate reduction curve (described in detail below) may be exploited at step 404 to determine the particular values for the VNS parameters needed to reduce patient heart rate below the tolerance threshold.

In this manner, VNS is continuously and chronically delivered to mitigate heart failure. The patient benefits from Mode 1 VNS (i.e. anti-HF therapy) due to its ability to restore proper autonomic balance and reduce cardiac inflammation. This effect is desired chronically. However, at times when the heart rate increases beyond the tolerance threshold, the device switches to Mode 2 to introduce controlled HR reduction along with anti-HF therapy. Regardless of whether Mode 1 or Mode 2 is employed, diagnostic data is preferably recorded at step 406 to specify, e.g., the current VNS Mode, the VNS parameters being used, the heart rate of the patient, etc., for subsequent clinician review during a follow-up session with the patient.

### Overview of Controlled Heart Rate Curve-Based VNS Techniques

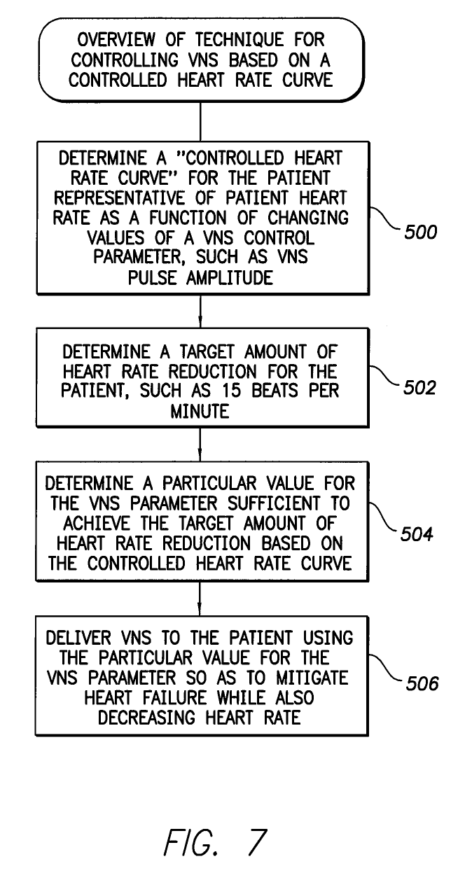

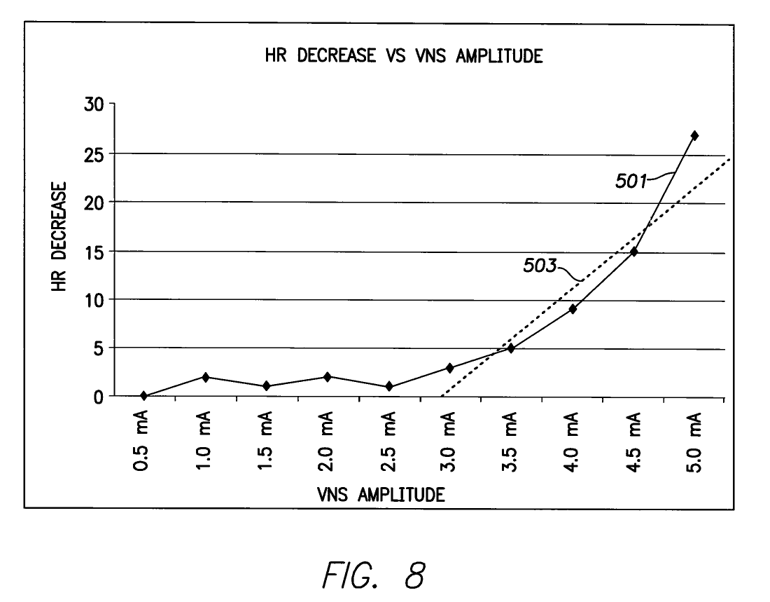

FIGS. 7 and 8 provide a broad overview of controlled heart rate curve-based VNS techniques that may be exploited by the pacer/ICD of FIG. 1 or other suitable VNS controller for achieving particular targeted levels of heart rate reduction (if any is needed) via VNS. Briefly, beginning at step 500, the pacer/ICD determines the “controlled heart rate curve” for the patient, which is representative of patient heart rate as a function of changing values of a selected VNS control parameter, such as VNS pulse amplitude. An exemplary controlled heart rate curve 501 is shown in FIG. 8 for VNS pulse amplitude. As can be seen, increasing pulse amplitude has no significant effect on heart rate within this patient until about 3.0 mA is reached, above which heart rate increases significantly due to increasing recruitment of Type C vagal fibers. The curve may be constructed (as will be explained more fully below) based on individual test values for the VNS parameter and the resulting heart rate reduction (if any). Otherwise conventional linear regression techniques can be used to fit a curve to the data points to yield the final controlled heart rate curve. In one example, as shown, a straight line 503 is fit to any data points where heart rate reduction is strongly affected by VNS amplitude. The slope of line 503 may then be used to easily convert target heart rate values to VNS pulse amplitudes, or vice versa.

At step 502 of FIG. 7, the pacer/ICD determines a target amount of heart rate reduction needed for the patient, such as a reduction of 15 beats per minute. This value may be determined, for example, based on the current heart rate of the patient relative to the above-described tolerance threshold. If the current rate exceeds the tolerance threshold by 15 bpm, then a reduction of at least 15 bpm is warranted. In any case, at step 504, the pacer/ICD determines a particular value for the VNS parameter sufficient to achieve the appropriate amount of heart rate reduction based on the controlled heart rate curve. For the example where a 15 bpm reduction is needed for a patient having the controlled heart rate curve of FIG. 8, a VNS pulse amplitude of 4.5 mA is thereby determined based on the curve. In circumstances where little or no heart rate reduction is needed, then the pacer/ICD preferably selects the highest value for the VNS parameter that is consistent with minimal heart rate reduction. For example, if the target amount of heart rate reduction is less than 3 bpm, then any pulse amplitude value in the range of 0.5 mA to 2.5 mA might potentially be selected based in the curve. The highest of these amplitude values is chosen so as to recruit the most Type A & B fibers to achieve heart failure mitigation.

Note that, as with the above-described heart rate reduction threshold, each VNS parameter generally has a different controlled heart rate curve. In use, the pacer/ICD is usually programmed to ascertain only the controlled heart rate curve for one particular VNS parameter, such as VNS pulse amplitude, which the pacer/ICD then adjusts to control VNS. However, in general, a controlled heart rate curve might be determined for any or all adjustable VNS control parameters including VNS pulse amplitude, pulse frequency, pulse width, pulse shape or any of a variety of VNS burst stimulation parameters. So long as the value of a given VNS parameter has some influence over the number of Type C vagal fiber that are recruited via VNS, then the parameter can have a corresponding controlled heart rate curve.

The general heart rate curve-based VNS techniques of FIG. 7 can be employed in connection with the threshold-based VNS techniques of FIGS. 2-6 to, e.g., achieve a target amount of heart rate reduction within Mode 2. However, the techniques of FIG. 7 can be employed separately, without necessarily specifying or quantifying any individual heart rate reduction thresholds.

### Exemplary Controlled Heart Rate Curve-Based Technique

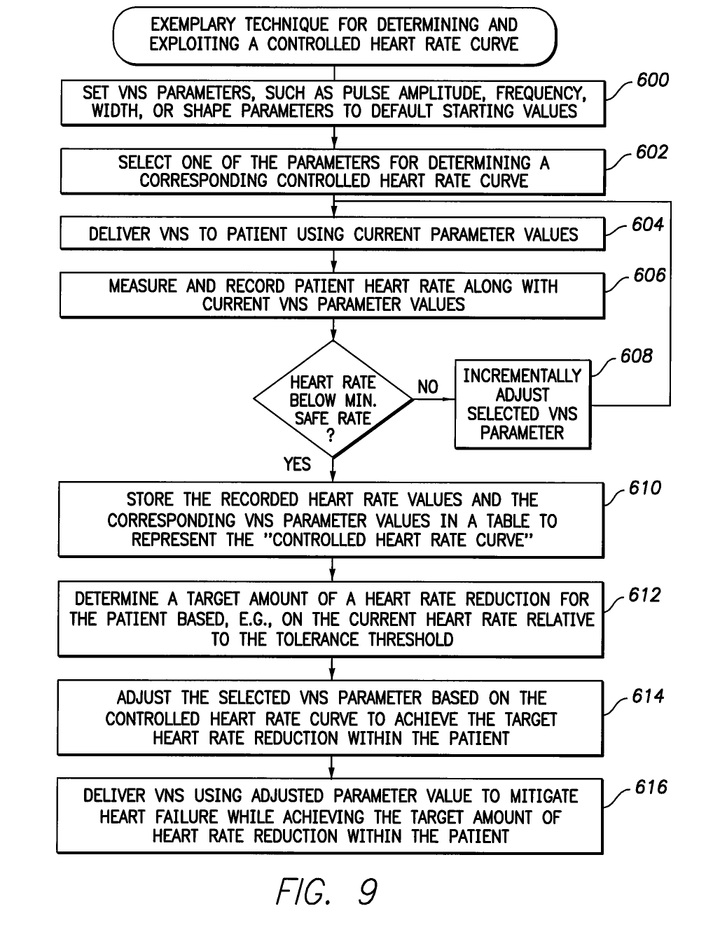

Turning now to FIG. 9, an exemplary technique for determining and exploiting controlled heart rate curves will be described. Beginning at step 600 of FIG. 3, the pacer/ICD sets the programmable VNS parameters (such as pulse amplitude, frequency, width, shape parameters or burst parameters) to default starting values. At step 602, the pacer/ICD then selects one of the parameters for determining a controlled heart rate reduction curve for that parameter. This selection may be based on pre-programming of the pacer/ICD.

At step 604, the pacer/ICD delivers VNS to the patient using the current VNS control parameter values and, at step 606, measures and records patient heart rate values along with the current VNS parameter values. So long as the heart rate does not fall below a minimum safe heart rate, the pacer/ICD incrementally adjusts the selected VNS parameter at step 608, while continuing to deliver VNS and while monitoring heart rate. The minimum safe heart rate is a pre-programmed value specified, e.g., by the clinician programming the device. It may be specified as a fixed heart rate value, such as 50 bpm, or may be specified as a reduction relative to the rest heart rate of the patient, such as a maximum reduction of 25 bpm below the rest rate. Each iteration of steps 604-608 can be set to, e.g., in the range of ten to thirty seconds to allow time for the VNS stimulation to achieve a stabilized heart rate. The average value of the stabilized heart rate may then be calculated. Note also that, as mentioned above, the actual heart rate of the patient need not be explicitly measured or calculated. R-R intervals can instead be used.

Once the minimum safe heart rate is reached, the pacer/ICD, at step 610, stores the recorded heart rate values and the corresponding VNS parameter values in a table to represent the controlled heart rate curve. As noted, linear regression may be used to fit a curve to the data. Thereafter, the controlled heart rate curve may be specified in terms of the coefficients of a best-fit equation. At step 612, the pacer/ICD then determines a target amount of a heart rate reduction for the patient (assuming a reduction is warranted). This may be determined, as noted, based on the current heart rate of the patient relative to the tolerance threshold for the patient so as to reduce the heart rate below the tolerance threshold. In any case, at step 614, the pacer/ICD adjusts the selected VNS parameter based on the controlled heart rate curve to achieve the target heart rate reduction within the patient. In the example already described with reference to FIG. 8, if a 15-bpm reduction is needed for the patient, the VNS pulse amplitude is thereby set to 4.5 mA to achieve the target reduction.

Further VNS is then delivered at step 614 using the adjusted VNS parameter value so as to mitigate heart failure while achieving the target heart rate reduction. Once further heart rate reduction is no longer needed, the VNS parameter may be reset to its initial default value or other suitable values.

Note that, similar to the embodiments discussed above, if the initially selected VNS parameter is iterated through its entire range of acceptable values without triggering any significant change in heart rate, then one of the other VNS parameters can instead be selected by the pacer/ICD for generating a controlled heart rate reduction curve for that parameter. If no combination of parameters is found that produces a suitable heart rate reduction curve, then warning signals may be generated to notify the clinician there might be a problem with the VNS stimulator within the patient. Also, note that the controlled heart rate curve determined at step 610 for a given VNS parameter can depend on the current values of the other VNS parameters, for the reasons already discussed by way of FIG. 4.

An exemplary algorithm for collecting the data for the controlled heart rate curve as a Test_HRVector is as follows:

```
  RRSafeMax = predetermined value.
  PreTestAverage = Collect & Average Next 10 R-R Intervals.
  For TestVoltage = 0.5 mA to 5 mA
    Initiate VNS Pacing @ TestVoltage.
    StepAverage = Collect & Average Next 10 R-R Intervals ‘If any R-R
            interval > RRSafeMax, exit test.
    Store {StepAverage, TestVoltage} in Test_HRVector.
  Increase TestVoltage by 0.5 mA
Terminate VNS Pacing.
```

Table I provides exemplary data collected using the algorithm:

TABLE I

| Amplitude | HR Decrease |
|---|---|
| 0.5 mA | 0 |
| 1.0 mA | 2 |
| 1.5 mA | 1 |
| 2.0 mA | 2 |
| 2.5 mA | 1 |
| 3.0 mA | 3 |
| 3.5 mA | 5 |
| 4.0 mA | 9 |
| 4.5 mA | 15 |
| 5.0 mA | 27 |

What have been described are various techniques for controlling VNS. For the sake of completeness, a detailed description of an exemplary pacer/ICD for performing these techniques will now be provided. However, principles of invention may be implemented within other pacer/ICD implementations or within other implantable devices such as stand-alone VNS devices. Furthermore, although examples described herein involve processing of VNS data by the implanted device itself, some operations may be performed using an external device, such as a bedside monitor, device programmer, computer server or other external system. For example, recorded heart rate reduction vs. VNS parameter data may be transmitted to the external device, which processes the data to determine heart rate reduction thresholds or to generate controlled heart rate reduction curves. Processing by the implanted device itself is preferred as that allows the device to update these thresholds and curves on-demand to respond to changes within the patient as might be brought on by changes in medication or the progression/regression of heart disease.

### Exemplary Pacemaker/ICD

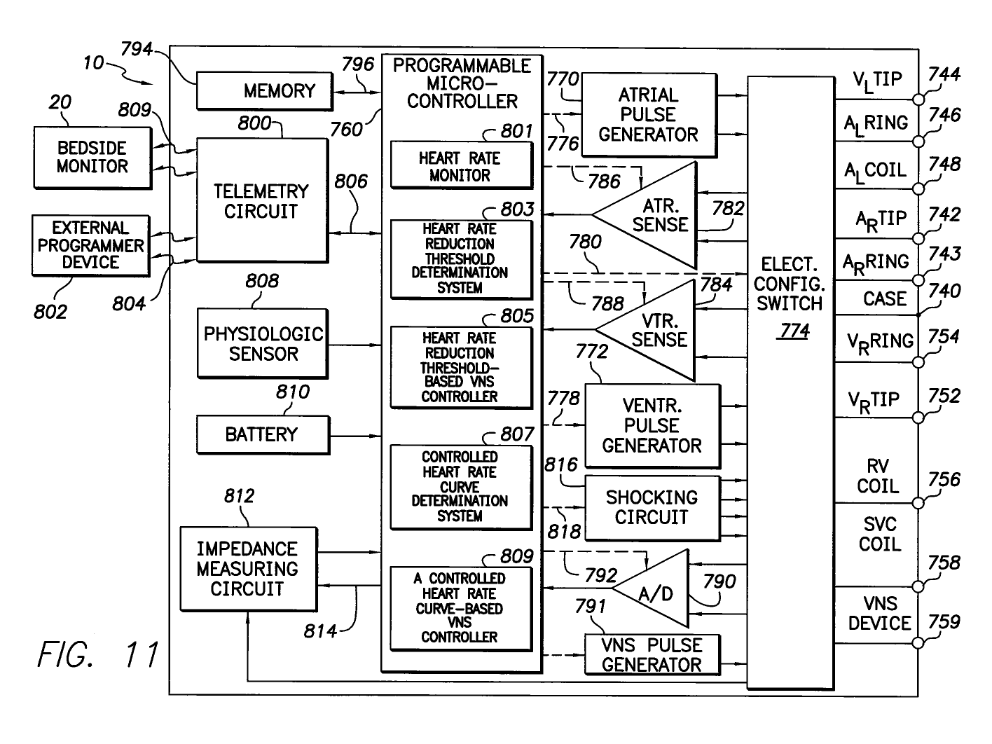

With reference to FIGS. 10 and 11, a description of an exemplary pacer/ICD will now be provided. FIG. 10 provides a simplified block diagram of the pacer/ICD, which is a dual-chamber stimulation device capable of treating both fast and slow arrhythmias with stimulation therapy, including cardioversion, defibrillation, pacing stimulation, as well as for controlling VNS. To provide atrial chamber pacing stimulation and sensing, pacer/ICD 710 is shown in electrical communication with a heart 712 by way of a left atrial lead 720 having an atrial tip electrode 722 and an atrial ring electrode 723 implanted in the atrial appendage. Pacer/ICD 710 is also in electrical communication with the heart by way of a right ventricular lead 730 having, in this embodiment, a ventricular tip electrode 732, a right ventricular ring electrode 734, a right ventricular (RV) coil electrode 736, and a superior vena cava (SVC) coil electrode 738. Typically, the right ventricular lead 730 is transvenously inserted into the heart so as to place the RV coil electrode 736 in the right ventricular apex, and the SVC coil electrode 738 in the superior vena cava. Accordingly, the right ventricular lead is capable of receiving cardiac signals, and delivering stimulation in the form of pacing and shock therapy to the right ventricle.

To sense left atrial and ventricular cardiac signals and to provide left chamber pacing therapy, pacer/ICD 710 is coupled to a CS lead 724 designed for placement in the “CS region” via the CS os for positioning a distal electrode adjacent to the left ventricle and/or additional electrode(s) adjacent to the left atrium. As used herein, the phrase “CS region” refers to the venous vasculature of the left ventricle, including any portion of the CS, great cardiac vein, left marginal vein, left posterior ventricular vein, middle cardiac vein, and/or small cardiac vein or any other cardiac vein accessible by the CS. Accordingly, an exemplary CS lead 724 is designed to receive atrial and ventricular cardiac signals and to deliver left ventricular pacing therapy using at least a left ventricular tip electrode 726, left atrial pacing therapy using at least a left atrial ring electrode 727, and shocking therapy using at least a left atrial coil electrode 728. With this configuration, biventricular pacing can be performed. Although only three pacing/sensing/shocking leads are shown in FIG. 10, it should also be understood that additional stimulation leads (with one or more pacing, sensing and/or shocking electrodes) might be used in order to efficiently and effectively provide pacing stimulation to the left side of the heart or atrial cardioversion and/or defibrillation.

To provide for VNS, pacer/ICD is coupled to a VNS lead 16 for stimulating the vagus nerve 12 via a vagal nerve stimulator 14. As with FIG. 1, a stylized representation of the vagus nerve and the vagal nerve stimulator are shown. Further information regarding the actual shape and location of the vagus nerve and suitable vagus nerve stimulators may be found in the above-cited patents, other VNS patents, or in the medical literature.

A simplified block diagram of internal components of pacer/ICD 710 is shown in FIG. 11. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation as well as providing for the aforementioned VNS therapy.

The housing 740 for pacer/ICD 710, shown schematically in FIG. 11, is often referred to as the “can”, “case” or “case electrode” and may be programmably selected to act as the return electrode for all “unipolar” modes. The housing 740 may further be used as a return electrode alone or in combination with one or more of the coil electrodes, 728, 736 and 738, for shocking purposes. The housing 740 further includes a connector (not shown) having a plurality of terminals, 742, 743, 744, 746, 748, 752, 754, 756, 758 and 759 (shown schematically and, for convenience, the names of the electrodes to which they are connected are shown next to the terminals). As such, to achieve right atrial sensing and pacing, the connector includes at least a right atrial tip terminal (A_{R} TIP) 742 adapted for connection to the atrial tip electrode 722 and a right atrial ring (A_{R} RING) electrode 743 adapted for connection to right atrial ring electrode 723. To achieve left chamber sensing, pacing and shocking, the connector includes at least a left ventricular tip terminal (V_{L} TIP) 744, a left atrial ring terminal (A_{L} RING) 746, and a left atrial shocking terminal (A_{L} COIL) 748, which are adapted for connection to the left ventricular ring electrode 726, the left atrial ring electrode 727, and the left atrial coil electrode 728, respectively. To Support right chamber sensing, pacing and shocking, the connector further includes a right ventricular tip terminal (V_{R} TIP) 752, a right ventricular ring terminal (V_{R} RING) 754, a right ventricular shocking terminal (V_{R} COIL) 756, and an SVC shocking terminal (SVC COIL) 758, which are adapted for connection to the right ventricular tip electrode 732, right ventricular ring electrode 734, the V_{R} coil electrode 736, and the SVC coil electrode 738, respectively. To support the VNS device, one or more VNS electrodes 759 are provided.

At the core of pacer/ICD 710 is a programmable microcontroller 760, which controls the various modes of stimulation therapy. As is well known in the art, the microcontroller 760 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 760 includes the ability to process or monitor input signals (data) as controlled by a program code stored in a designated block of memory. The details of the design and operation of the microcontroller 760 are not critical to the invention. Rather, any suitable microcontroller 760 may be used that carries out the functions described herein. The use of microprocessor-based control circuits for performing timing and data analysis functions are well known in the art.

As shown in FIG. 11, an atrial pulse generator 770 and a ventricular pulse generator 772 generate pacing stimulation pulses for delivery by the right atrial lead 720, the right ventricular lead 730, the CS lead 724 and/or the VNS lead via an electrode configuration switch 774. It is understood that in order to provide stimulation therapy in each of the four chambers of the heart, the atrial and ventricular pulse generators 770, 772 may include dedicated, independent pulse generators, multiplexed pulse generators or shared pulse generators. The pulse generators 770, 772 are controlled by the microcontroller 760 via appropriate control signals 776, 778, respectively, to trigger or inhibit the stimulation pulses. A VNS pulse stimulator 791 is also shown.

The microcontroller 760 further includes timing control circuitry (not separately shown) used to control the timing of such stimulation pulses (e.g., pacing rate, AV delay, atrial interconduction (inter-atrial) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 774 includes a plurality of switches for connecting the desired electrodes to the appropriate I/O circuits, thereby providing complete electrode programmability. Accordingly, the switch 774, in response to a control signal 780 from the microcontroller 760, determines the polarity of the stimulation pulses (e.g., unipolar, bipolar, combipolar, etc.) by selectively closing the appropriate combination of switches (not shown) as is known in the art.

Atrial sensing circuits 782 and ventricular sensing circuits 784 may also be selectively coupled to the right atrial lead 720, CS lead 724, and the right ventricular lead 730, through the switch 774 for detecting the presence of cardiac activity in each of the four chambers of the heart. Accordingly, the atrial and ventricular sensing circuits 782, 784 may include dedicated sense amplifiers, multiplexed amplifiers or shared amplifiers. The switch 774 determines the “sensing polarity” of the cardiac signal by selectively closing the appropriate switches, as is also known in the art. In this way, the clinician may program the sensing polarity independent of the stimulation polarity. Each sensing circuit 782, 784 preferably employs one or more low power, precision amplifiers with programmable gain and/or automatic gain control and/or automatic sensitivity control, bandpass filtering, and a threshold detection circuit, as known in the art, to selectively sense the cardiac signal of interest. The automatic gain/sensitivity control enables pacer/ICD 710 to deal effectively with the difficult problem of sensing the low amplitude signal characteristics of atrial or ventricular fibrillation. The outputs of the atrial and ventricular sensing circuits 782, 784 are connected to the microcontroller 760 which, in turn, are able to trigger or inhibit the atrial and ventricular pulse generators 770, 772 respectively, in a demand fashion in response to the absence or presence of cardiac activity in the appropriate chambers of the heart.

For arrhythmia detection, pacer/ICD 710 utilizes the atrial and ventricular sensing circuits 782, 784 to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “F-waves” or “Fib-waves”) are then classified by the microcontroller 760 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 790. The data acquisition system 790 is configured to acquire intracardiac electrogram signals, convert the raw analog data into a digital signal, and store the digital signals for later processing and/or telemetric transmission to an external device 802. The data acquisition system 790 is coupled to the right atrial lead 720, the CS lead 724, and the right ventricular lead 730 through the switch 774 to sample cardiac signals across any pair of desired electrodes. The microcontroller 760 is further coupled to a memory 794 by a suitable data/address bus 796, wherein the programmable operating parameters used by the microcontroller 760 are stored and modified, as required, in order to customize the operation of pacer/ICD 710 to suit the needs of a particular patient. Such operating parameters define, for example, pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate, as well as the aforementioned VNS parameters.

Advantageously, the operating parameters of the implantable pacer/ICD 710 may be non-invasively programmed into the memory 794 through a telemetry circuit 800 in telemetric communication with the external device 802, such as a programmer, transtelephonic transceiver or a diagnostic system analyzer. The telemetry circuit 800 is activated by the microcontroller by a control signal 806. The telemetry circuit 800 advantageously allows intracardiac electrograms and status information relating to the operation of pacer/ICD 710 (as contained in the microcontroller 760 or memory 794) to be sent to the external device 802 through an established communication link 804. Pacer/ICD 710 further includes an accelerometer or other physiologic sensor 808, commonly referred to as a “rate-responsive” sensor because it is typically used to adjust pacing stimulation rate according to the exercise state of the patient. However, the physiological sensor 808 may further be used to detect changes in cardiac output, changes in the physiological condition of the heart, or diurnal changes in activity (e.g., detecting sleep and wake states) and to detect arousal from sleep. Accordingly, the microcontroller 760 responds by adjusting the various pacing parameters (such as rate, AV delay, V-V delay, etc.) at which the atrial and ventricular pulse generators 770, 772 generate stimulation pulses. While shown as being included within pacer/ICD 710, it is to be understood that the physiologic sensor 808 may also be external to pacer/ICD 710, yet still be implanted within or carried by the patient. A common type of rate responsive sensor is an activity sensor incorporating an accelerometer or a piezoelectric crystal, which is mounted within the housing 740 of pacer/ICD 710. Other types of physiologic sensors are also known, for example, sensors that sense the oxygen content of blood, respiration rate and/or minute ventilation, pH of blood, ventricular gradient, etc.

The pacer/ICD additionally includes a battery 810, which provides operating power to all of the circuits shown in FIG. 11. The battery 810 may vary depending on the capabilities of pacer/ICD 710. For pacer/ICD 710, which employs shocking therapy, the battery 810 should be capable of operating at low current drains for long periods, and then be capable of providing high-current pulses (for capacitor charging) when the patient requires a shock pulse. The battery 810 must also have a predictable discharge characteristic so that elective replacement time can be detected. Accordingly, pacer/ICD 710 is preferably capable of high voltage therapy and appropriate batteries.

As further shown in FIG. 11, pacer/ICD 710 is shown as having an impedance measuring circuit 812 which is enabled by the microcontroller 760 via a control signal 814. Exemplary uses for the impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; and detecting the opening of heart valves, etc. The impedance measuring circuit 120 is advantageously coupled to the switch 74 so that any desired electrode may be used.

In the case where pacer/ICD 710 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 760 further controls a shocking circuit 816 by way of a control signal 818. The shocking circuit 816 generates shocking pulses of low (up to 0.5 joules), moderate (0.5-10 joules) or high energy (11 to 40 or more joules), as controlled by the microcontroller 760. Such shocking pulses are applied to the heart of the patient through at least two shocking electrodes, and as shown in this embodiment, selected from the left atrial coil electrode 728, the RV coil electrode 736, and/or the SVC coil electrode 738. The housing 740 may act as an active electrode in combination with the RV electrode 736, or as part of a split electrical vector using the SVC coil electrode 738 or the left atrial coil electrode 728 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 8-40 or more joules), delivered asynchronously (since R-waves may be too disorganized), and pertaining exclusively to the treatment of fibrillation. Accordingly, the microcontroller 760 is capable of controlling the synchronous or asynchronous delivery of the shocking pulses.

Insofar as VNS control is concerned, the microcontroller includes a heart rate monitor 801 and a heart rate reduction threshold determination system 803, which is operative to determine one or more heart rate reduction thresholds as already described with reference to FIG. 2. A heart rate reduction threshold-based VNS controller 805 controls VNS based, in part, on the heart rate reduction thresholds so to, e.g., deliver VNS to mitigate heart failure without also reducing heart rate, as already described with reference to FIG. 3. A controlled heart rate curve determination system 807 determines one or more controlled heart rate curves for the patient, as already described with reference to FIG. 7. A controlled heart rate curve-based VNS controller 809 controls VNS based, in part, on the controlled heart rate curve so to, e.g., achieve a target reduction in heart rate, as already described with reference to FIG. 9.

Depending upon the implementation, the various components of the microcontroller may be implemented as separate software modules or the modules may be combined to permit a single module to perform multiple functions. In addition, although shown as being components of the microcontroller, some or all of these components may be implemented separately from the microcontroller, using application specific integrated circuits (ASICs) or the like.

The principles of the invention may be exploiting using other implantable systems or in accordance with other techniques. Thus, while the invention has been described with reference to particular exemplary embodiments, modifications can be made thereto without departing from scope of the invention. Note that the term “including” as used herein is intended to be inclusive, i.e. “including but not limited to.”

What is claimed is:

1. A method for use with an implantable medical device for implant within a patient, the method comprising:
stimulating the vagus nerve of the patient in accordance with at least one adjustable vagus nerve stimulation (VNS) parameter while monitoring patient heart rate;
determining a heart rate reduction threshold level for the VNS parameter at which the stimulation begins to reduce heart rate; and
controlling further VNS based on the detected heart rate reduction threshold level.

2. The method of claim 1 wherein the VNS parameter includes one or more of stimulation pulse amplitude, width, frequency and shape.

3. The method of claim 1 wherein determining the heart rate reduction threshold level for the VNS parameter includes incrementally adjusting the VNS parameter until, at least, a predetermined minimum heart rate reduction level is detected.

4. The method of claim 3 wherein the predetermined minimum heart rate reduction level is in the range of two to three beats per minute (bpm).

5. The method of claim 3 wherein incrementally adjusting the VNS parameter is terminated if the patient's heart rate falls below a predetermined minimum acceptable heart rate threshold.

6. The method of claim 1 wherein controlling further VNS is performed to deliver VNS without incurring any significant reduction in patient heart rate.

7. The method of claim 6 wherein controlling further VNS to deliver VNS without incurring any significant reduction in heart rate is performed to trigger only Type A and Type B vagus nerve fibers.

8. The method of claim 6 wherein controlling further VNS is performed by delivering VNS pulses at a predetermined percentage of the heart rate reduction threshold level.

9. The method of claim 8 wherein the predetermined percentage is in the range of 25-95%.

10. The method of claim 1 wherein controlling further VNS is performed to achieve a reduction in patient heart rate.

11. The method of claim 10 wherein controlling further VNS to achieve a reduction in heart rate reduction is performed to trigger Type C vagus nerve fibers along with Type A and Type B vagus nerve fibers.

12. The method of claim 10 wherein controlling further VNS is performed by delivering VNS pulses at a predetermined level above the heart rate reduction threshold level.

13. The method of claim 12 wherein controlling further VNS to achieve a reduction in heart rate includes:
determining a controlled heart rate curve for the patient representative of heart rate decrease as a function of increasing values of the VNS parameter;
determining a preferred amount of heart rate reduction for the patient;
determining a particular value for the VNS parameter sufficient to achieve the preferred amount of heart rate reduction based on the controlled heart rate curve; and
delivering further VNS using the particular value for the VNS parameter.

14. The method of claim 13 wherein controlling further VNS to achieve a reduction in heart rate is performed in response to the patient's heart rate exceeding a predetermined tolerance threshold.

15. A system for use with an implantable medical device for implant within a patient, the system comprising:
a vagus nerve stimulation (VNS) device operative to deliver VNS to the heart of the patient in accordance with at least one adjustable VNS parameter;
a heart rate monitor;
a heart rate reduction threshold determination system operative to determine a threshold level for a selected VNS parameter at which VNS begins to reduce heart rate as measured by the heart rate monitor; and
a VNS controller operative to control further VNS based on the heart rate reduction threshold.

16. The system of claim 15 further including a controlled heart rate curve determination unit operative to determine a controlled heart rate curve for the patient representative of heart rate decrease within the patient as a function of increasing values of the VNS parameter.

17. The system of claim 15 wherein the VNS controller is further operative to control VNS based on the controlled heart rate curve.

18. A system for use with an implantable medical device for implant within a patient, the method comprising:
means for stimulating the vagus nerve in accordance with at least one vagus nerve stimulation (VNS) parameter while monitoring heart rate;
means for determining a threshold level for the VNS parameter at which the stimulation begins to reduce heart rate; and
means for controlling further VNS based on the detected threshold level.

19. A method for use with an implantable medical device for implant within a patient wherein the device is equipped to stimulate the vagus nerve of the patient in accordance with at least one adjustable vagus nerve stimulation (VNS) parameter, the method comprising:
determining a controlled heart rate curve for the patient representative of heart rate decrease within the patient as a function of increasing values of the VNS parameter;
determining a preferred amount of heart rate reduction for the patient;
determining a particular value for the VNS parameter sufficient to achieve the preferred amount of heart rate reduction based on the controlled heart rate curve; and
delivering VNS to the patient using the particular value for the VNS parameter.

20. A system for use with an implantable medical device for implant within a patient, the system comprising:
a vagus nerve stimulation (VNS) device operative to deliver VNS to the heart of the patient in accordance with at least one adjustable VNS parameter;
a heart rate monitor; and
a controlled heart rate curve determination system operative to determine a controlled heart rate curve for the patient representative of heart rate decrease within the patient as a function of increasing values of the adjustable VNS parameter; and
a VNS controller operative to control deliver of VNS to the patient based on the controlled heart rate curve.
